(19) United States
(12) Patent Application Publication
(10) Pub. No.: US 2004/0215074 A1
(43) Pub. Date: Oct. 28, 2004

(54) Imaging and scoring method for cervical spinal impairment using magnetic resonance imaging

(76) Inventors: Vincenzo Giuliano, Winter Springs, FL (US); Concetta Giuliano, Winter Springs, FL (US)

Correspondence Address: EDWARD M. LIVINGSTON, PA, 963 TRAIL TERRACE DRIVE, NAPLES, FL, 34103, US

(21) Appl. No.: 10/420,983

(22) Filed: Apr. 24, 2003

(51) Int. Cl. A61B 5/055

(52) U.S. Cl. 600/415

(57) ABSTRACT

A non-evasive scoring method uses magnetic resonance imaging following kinematic movement of the cervical spine to determine the degree of impairment following injury, particularly soft tissue injury typical in rear, low-impact motor vehicle collisions. A system using the method includes an assessment algorithm employing four (4) categories of variables of hypolordosis, motion restriction, disk herniation and spinal stenosis, a numerical score assigned to each variable and a clinical classification assigned to a patient based on summation of scores assigned to each variable. This method is designated by the acronym, STIP, for Soft Tissue Injury Protocol.

### BACKGROUND OF THE INVENTION

The present invention relates generally to a non-invasive method for determining the kinematic function of the cervical spine using magnetic resonance imaging. More particularly, the invention relates to an imaging protocol, clinical algorithm, and scoring system to determine the degree of impairment from hyperflexion/hyperextension injuries of the cervical spine resulting from rear, low-impact motor vehicle collisions, as a basis to monitor therapeutic measures prescribed in the clinical management such neck injuries.

Cervical spine trauma is common following rapid acceleration or deceleration during low-impact motor vehicle collisions, resulting in more than three million injuries annually in the United States. The cervical spine is particularly susceptible to such injury because of its wide range of motion and relative absence of supporting structures. Post-mortem studies of injured subjects have isolated these injuries to the posterior cervical complex, joint capsule, interspinous/supraspinous ligaments, and posterior portion of the annulus fibrous, which are occult to conventional imaging methods. Various non-invasive kinematic methods have been devised to establish differences in the kinematic function of the cervical spine between normal and injured subjects, but such methods do not adequately address soft tissue damage. Computerized tomography (CT Scan) and magnetic resonance imaging (MRI Scan) address soft tissue damage, but do not assess kinematic movement or function. Assessment of both soft tissue damage and kinematic function have important implications medically and legally in order to determine not only the extent of injury, but also the effects of manipulative resonance imaging.

The most pertinent prior art includes the following patents:

| Pat. No.: | Inventor: | Issue Date: |
|---|---|---|
| 5,154,178 | Shah | Oct. 13, 1992 |
| 5,203,346 | Fuhr et al. | Apr. 20, 1993 |
| 5,349,956 | Bonutti | Sep. 27, 1994 |
| 6,473,717 | Claussen et al. | Oct. 29, 2002 |

Contrary to the prior art, the present invention provides the only systematic method that can be applied to normal and injured patients for evaluating soft tissue injury and kinematic function to the cervical spine in a single combined test using magnetic resonance imaging.

### SUMMARY OF THE INVENTION

An object of the present invention is to provide a reliable diagnostic method for evaluating the anatomic and functional basis for injury in the cervical spine using magnetic resonance imaging.

Another object of the present invention is to provide a useful clinical algorithm and scoring method to establish the degree of impairment to differentiate the cervical spines of injured versus non-injured patients.

The present invention fulfills the above and other objects by providing a non-evasive method and system for determining the kinematic function of the cervical spine which includes an assessment algorithm, a numerical scoring system, and a classification system. The assessment algorithm encompasses variables related to anatomic and kinetic function and various subcategories including cervical hypolordosis, motion restriction, disk herniation, and spinal stenosis. A numerical scoring system includes a scoring system for each variable and a classification system of five (5) classes into which a patient is placed based on a summation of the scores assigned to each variable according to degree of impairment which serves as a basis for treatment. The actual method involved includes positioning a neck injury patient in the center of a magnetic resonance imaging (MRI) coil so as to prevent movements in a forward and backward direction to approximate the physiological motion pattern of the cervical spine. Then, two (2) increments of physiological movements, one (1) to flexion and one (1) to extension are conducted. The resulting MRI images are then processed and viewed so that the kinematic function of the cervical spine can be assessed according to the subcategories and variables in the system referenced above depending on the degree of the presence of each variable. The scores are then totaled and used to assign the patient to one of five (5) classes according to the degree of pain and impairment.

As the present invention has particular applicability to soft tissue injuries, it has been labeled the Soft Tissue Injury Protocol (STIP) and is hereinafter referred to as the STIP.

The above and other objects, features and advantages of the present invention should become even more readily apparent to those skilled in the art upon a reading of the following detailed description in conjunction with the drawings wherein there is described and shown preferred embodiments of the invention.

### BRIEF DESCRIPTION OF THE DRAWINGS

In the following detailed description, reference will be made to the attached drawings in which:

FIG. 1 is a chart of subcategories of variables used in the STIP scoring method and system; and

FIG. 2 is a chart of the classification system employed in the STIP scoring method and system.

### DESCRIPTION OF THE PREFERRED EMBODIMENTS

For imaging purposes, rapid T2-weighted magnetic resonance imaging is utilized as the principal technology in the present invention because of its ability to distinguish aqueous structures, such as the nucleus pulposus and cerebrospinal fluid, intrinsic to the optimal delineation of diskoligamentous soft tissue injuries. A rapid spin-echo T2-weighted pulse sequence enables sufficient signal-to-noise and resolution, thin scan sections, and rapid imaging capability necessary to visualize the spinal cord, intervertebral disks, ligaments, apophyseal joints, and spinous processes, all in a single acquisition.

To perform the STIP scoring method, a patient's neck is positioned in the center of a suitable imaging coil, in combination with a magnetic resonance imaging system. Since spinal movements are not assisted, no additional apparatus is required, as is required by other methods in the prior art. The STIP scoring method uses supplies and accessories available to most state-of-the-art magnetic resonance imaging systems. Coil selection is restricted only by its diameter in the anteroposterior dimension, so as not to restrict the normal physiologic motion of the normal patient cervical spine. It is also imperative to ensure that the cervical spine shows no evidence of fracture, listhesis, spondylosis, or previous surgery.

Clinical subjects must be twelve (12) weeks post injury, to eliminate the possibility of muscle spasm resulting in a false positive result. Scan parameters must conform with guidelines recognized by The American College of Radiology, as minimum standards of quality established by the present art. Suggested scan parameters for a rapid T-2-weighted fast-recovery, fast-spin echo sequence are: repetition time to echo time (in milliseconds) of 2,600/108, echo train of 16; number of excitations of two (2); slice thickness of five (5) mm; and total imaging time of ninety (90) seconds per acquisition.

Spinal movements are initiated under direct medical supervision but are unassisted, so as to permit physiologic movements and not potentiate the risk of injury. Two (2) acquisitions are performed, one following full flexion; the second, following full extension, although the two (2) movements are interchangeable. Images are formatted on a viewing screen frame-per-frame and evaluated for cervical lordosis, segmental spinal motion (particularly the herniation, and degree of spinal stenosis. Quantitative assessment of cervical kinematic spinal motion also can be applied to monitor kinematic function useful to those skilled in the art in order to determine the efficacy of medical and manipulative therapy.

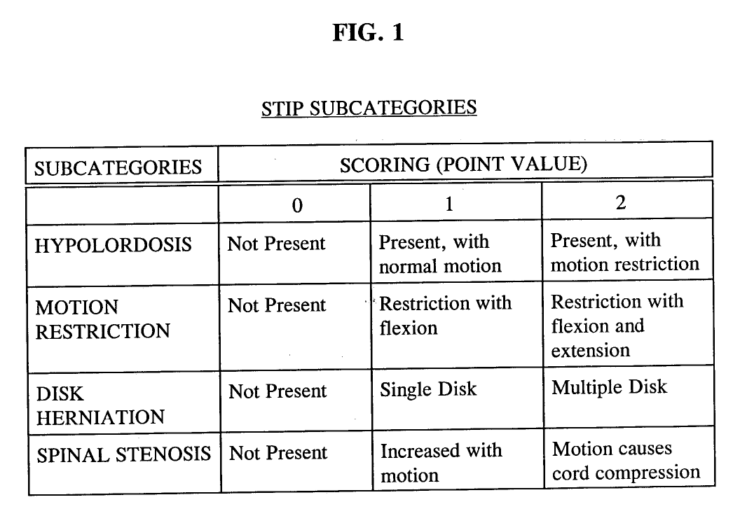

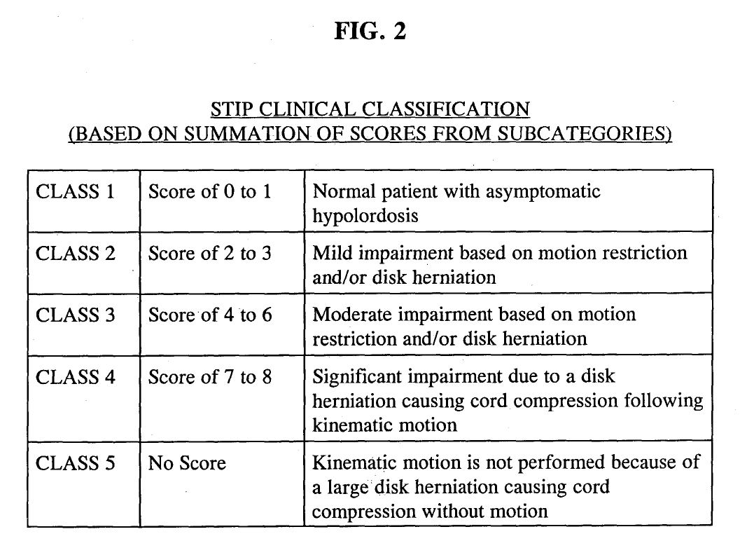

The present invention establishes distinct differences between normal and injured patients based on mechanisms of injury well known in the present art. During the first phase of hyperflexion/perextension injury caused by rear, low-impact collisions, the cervical spine forms an S-shaped curve, with hyperflexion of the upper cervical segments and simultaneous hyperextension of the lower cervical segments. During the second phase, all spinal segments become hyperextended. Hyperflexion produces the greatest stress and potential for injury to the posterior cervical complex of the mid cervical spine, the fulcrum point. The posterior cervical complex consists of the posterior longitudinal ligament, joint capsule, interspinous/supraspinous ligaments and ligenta flava. These injuries evade detection by magnetic resonance imaging when kinematic motion methods are not applied, as in the present art. Both spinal motion and soft tissue injury are evaluated simultaneously using the present invention. Injured patients demonstrate hypolordosis and abnormal biochemical cervical kinematic motion, with segmental motion restriction and fixation, particularly the fan-like movements of the spinous processes. In cases where disk herniations are also observed, the extradural impression causing spinal stenosis changes with positional movements, providing a distinct advantage over conventional magnetic resonance imaging examinations performed in the neutral/supine position. The new clinical insights provided by the present invention permit a clinical algorithm and scoring system to be applied in the clinical management of patients in the subacute setting, summarized in the tables of FIGS. 1 and 2.

By reference to the drawings, the following examples are intended to further illustrate the invention described herein.

### EXAMPLE 1

A normal subject, not afflicted by hyperflexion/perextension injury, and showing no clinical signs and symptoms, demonstrates, following application of the STIP scoring method, normal cervical lordosis, normal segmental kinematic motion following flexion and extension, no disk herniation, and no spinal stenosis. Quantitative cervical kinematic motion is approximated as 50 degrees flexion and 60 degrees extension, within normal limits established by the prior art. Thus, the STIP score is 0 and the STIP Clinical Classification is Class 1, consistent with no clinical impairment. The clinical conclusion is that no clinical impairment is seen and that no treatment is necessary.

### EXAMPLE 2

An injured patient is involved in a rear, low-impact motor vehicle collision and demonstrates positive signs and symptoms at twelve (12) weeks following injury. Plain radiographs (x-rays) of the cervical spine are negative. Following the scoring using point values in FIG. 1, the STIP scoring method is applied and shows hypolordosis with motion restriction, thus yielding a subscore of 2. Segmental spinal motion is restricted in both flexion and extension for a subscore of 2. A single small central disk herniation is noted at C5/C6 for a subscore of 1, resulting in mild extradural impression which increases the degree of spinal stenosis with flexion, but does not result in spinal cord compression for a subscore of 1. Quantitative cervical kinematic motion is approximated as 25 degrees flexion and 35 degrees extension, below normal limits established by the prior art. For the patient in this example, the total STIP score is 6 and therefore, the STIP Clinical Classification is Class 3, consistent with moderate clinical impairment. The clinical conclusion is that moderate clinical impairment is seen and that treatment is indicated on this basis.

Following a standard twelve (12) week treatment regimen, consisting of medical and manipulative therapy, the subject shows subjective improvement of clinical signs and symptoms. The STIP scoring method is again applied at twenty-four (24) weeks post injury. Hypolordosis is still present with motion restriction for a subscore of 2. Segmental spinal motion is overall improved but a mild residual restriction is noted following flexion, but extension is within normal limits for a subscore of 1. The small disk herniation seen previously is not present for a subscore of 0. Finally, no spinal stenosis is noted for a subscore of 0. Quantitative cervical kinematic motion is approximated as 35 degrees flexion and 50 degrees extension; flexion is still slightly below the normal limit. The total STIP score for this patient is now 3 and the STIP clinical classification is Class 2, consistent with mild clinical impairment. The clinical conclusion is that mild residual clinical impairment is seen, with interval improvement following medical and manipulative therapy, and that maximum medical improvement, commonly referred to as the MMI, has been achieved.

### EXAMPLE 3

The injured patient is involved in a rear, low-impact motor vehicle collision and demonstrates delayed signs of radiculopathy and upper extremity weakness at twelve (12) weeks following injury, despite medical and manipulative therapy, and normal plain radiographs (x-rays) of the cervical spine. The STIP scoring method is applied and shows hypolordosis with significant segmental motion restriction for a subscore of 2 in both flexion and extension yielding a subscore of 2. A large central disk herniation is seen at C4/C5, resulting in significant spinal stenosis and impingement of the spinal cord following flexion. A small central disk herniation is also noted at C5/C6, without cord impingement, but with mildly increased spinal stenosis following flexion. The multiple disk herniation results in a subscore of 2 and the spinal stenosis with cord impairment a subscore of 2. Quantitative cervical kinematic motion is approximated as 10 degrees flexion and 25 degrees extension, well below normal limits established by the prior art. Thus, in this example the STIP score is 8 and the STIP Clinical Classification is Class 4, consistent with significant clinical impairment. The clinical conclusion is that significant clinical impairment is seen and that intervention is indicated to prevent spinal cord injury.

Surgical intervention was performed at fourteen (14) weeks post injury, consisting of anterior cervical diskectomy and fusion, with complete resolution of clinical signs and symptoms. A follow-up evaluation using the STIP method at twenty-six (26) weeks post injury (12 weeks following surgery) shows no residual or recurrent disk herniation or a subscore of 0. Hypolordosis is present, with a mild residual restriction (a subscore of 2) in segmental spinal motion in the mid cervical spine following flexion for a subscore of −1. Extension is within normal limits. Quantitative cervical kinematic motion is approximated as 35 degrees flexion and 50 degrees extension. Thus, this patient's STIP score is now 3 and the STIP Clinical Classification is Class 2, consistent with mild clinical impairment. The clinical conclusion is that maximum medical improvement has been achieved, and that no further intervention is indicated on this basis.

While the present invention is described in conjunction with specific preferred embodiments and procedures, it should be understood that it may be embodied in other specific forms or variations without departing from its spirit or essential characteristics. For instance, future technological advancements in magnetic resonance imaging systems may produce images with greater resolution and speed than is currently available in the present invention. Accordingly, the embodiments described above are to be considered in all respects as illustrative and not restrictive, the scope of the invention being indicate by the appended claims rather than the foregoing description, and that all changes which come within the meaning and range of equivalency of the claims to be embraced therein.

What is claimed is:

1. A non-evasive method, using magnetic resonance imaging (MRI), for determining a kinematic function of a cervical spine, comprising the steps of:
a. positioning a neck of a patient in the center of a suitable magnetic resonance imaging coil so as to permit movements in a forward and backward direction approximating a physiologic motion pattern of the cervical spine;
b. initiating unassisted physiologic movements, first to flexion, and then to full extension;
c. optimizing imaging parameters to yield MRI scan images;
d. processing the images for viewing; and
e. assessing the kinematic function of the cervical spine during maximum flexion and maximum extension.

2. The method of claim 1 wherein accessing encompasses variables related to anatomic and kinematic function, with sub-categories comprising:
a. Cervical hypolordosis;
b. Motion restriction;
c. Disk herniation; and
d. Spinal stenosis.

3. The method of claim 2 further comprising assigning a numerical score to each of the subcategories, said scoring comprising:
a. for hypolordosis, a score of 0, if not present; a score of 1, if present with normal motion; a score of 2, if present with motion restriction;
b. for motion restriction, a score of 0, if not present; a score of 1, if motion restriction is only with flexion; a score of 2, if motion restriction is both with flexion and extension;
c. for disk herniation, a score of 0, if not present; a score of 1, if present at a single cervical spine level; a score of 2, if present at more than one (1) cervical spine level; and
d. for spinal stenosis, a score of 0, if not present; a score of 1, if stenosis is increased by a disk herniation with motion; a score of 2, if the spinal cord is compressed by a disk herniation with motion.

4. The method of claim 3 further comprising classifying, the degree of impairment of kinematic movement and function of the cervical spine derived from the summation of scores according to claim 3 in to one (1) of five (5) classes comprising of:
a. Class 1, a score of 0 to 1, designating the normal patient with asymptomatic hypolordosis or disk herniation;
b. Class 2, a score of 2 to 3, designating mild impairment, based on the degree of motion restriction and/or disk herniation;
c. Class 3, a score of 4 to 6, designating moderate impairment, based on the more significant degree of motion restriction and/or disk herniation;
d. Class 4, a score of 7 to 8, designating significant impairment, based on the presence of cord compression caused by a disk herniation following motion; and
e. Class 5, no score, a severe form of impairment due to a large disk herniation compressing the spinal cord without kinematic movement.

5. The system for determining a kinetic function of cervical spine using magnetic resonance imaging (MRI), said system comprising:
a. an assessment algorithm;
b. a numerical scoring system; and
c. a classification system.

6. The system of claim 5 wherein the assessment algorithm encompasses variables related to anatomic and kinetic function in the subcategories of cervical hypolordosis, motion restriction, disk herniation, and spinal stenosis.

7. The system of claim 5 wherein the numerical scoring system comprises assigning a score for each of the subcategories in the assessment algorithm.

8. The system of claim 6 wherein the numerical scoring system comprises assigning a score for each of the sub-categories in the assessment algorithm.

9. The system of claim 6 wherein the classification system for the degree of impairment in kinetic function of the cervical spine derived from the summation of scores for each of the variables comprises five (5) classes as follows:
a. Class 1, a score of 0 to 1, designating the normal patient with asymptomatic hypolordosis or disk herniation;
b. Class 2, a score of 2 to 3, designating mild impairment, based on the degree of motion restriction and/or disk herniation;
c. Class 3, a score of 4 to 6, designating moderate impairment, based on the more significant degree of motion restriction and/or disk herniation;
d. Class 4, a score of 7 to 8, designating significant impairment, based on the presence of cord compression caused by a disk herniation following motion; and
e. Class 5, no score, a severe form of impairment due to a large disk herniation compressing the spinal cord without kinematic movement.

10. The system of claim 7 wherein the classification system for the degree of impairment in kinetic function, the function of the cervical spine derived from the summation of scores for each of the variables comprises five (5) classes as follows:
a. Class 1, a score of 0 to 1, designating the normal patient with asymptomatic hypolordosis or disk herniation;
b. Class 2, a score of 2 to 3, designating mild impairment, based on the degree of motion restriction and/or disk herniation;
c. Class 3, a score of 4 to 6, designating moderate impairment, based on the more significant degree of motion restriction and/or disk herniation;
d. Class 4, a score of 7 to 8, designating significant impairment, based on the presence of cord compression caused by a disk herniation following motion; and
e. Class 5, no score, a severe form of impairment due to a large disk herniation compressing the spinal cord without kinematic movement.

11. The method of claim 1 wherein the MRI scan images are sagittal T2-weighted MRI scan images with a section thickness of 5 millimeters or less, and total imaging time of three (3) minutes or less.
